(19) United States
(12) Patent Application Publication
(10) Pub. No.: US 2011/0309242 A1
(43) Pub. Date: Dec. 22, 2011

(54) Radiation-activated Fiducial Markers for Organ Tracking

(75) Inventor: Jonathan S. Maltz, Oakland, CA (US)

(73) Assignee: SIEMENS MEDICAL SOLUTIONS USA, INC., Malvern, PA (US)

(21) Appl. No.: 12/816,927

(22) Filed: Jun. 16, 2010

(51) Int. Cl. G21H 5/02; A61N 5/10

(52) U.S. Cl. 250/252.1; 250/303; 250/393; 600/424

(57) ABSTRACT

A system may include transmission of an activation beam to one or more non-radioactive elements disposed in a patient volume, wherein the elements become radioactive in response to the activation beam, detection of radiation emitted by the one or more now-radioactive elements, determination of respective locations of the one or more now-radioactive elements based on the detected radiation, and transmission of a treatment beam to the patient volume based on the respective locations.

### BACKGROUND

The embodiments described below relate generally to systems for delivering radiation therapy. More specifically, some embodiments are directed to position verification systems used in conjunction with such delivery.

### DESCRIPTION

Conventional radiation therapy systems direct a beam of protons, x-ray photons, neutrons or positive ions to a target within a patient volume. The radiation dose delivered by the beam kills tissue cells within the target by damaging the DNA thereof.

Various systems are used to ensure that a target is located in the path of a beam and that sensitive structures are avoided in accordance with a treatment plan. Such systems may acquire a two-dimensional projection image along the axis of a beam in order to identify which internal structures are located in the beam path. The “beam's eye” view provided by such projection images identifies the relationship between the beam path and the internal structures, but only in directions perpendicular to the beam axis.

Several projection images, acquired at different rotational angles, may be combined to create a three-dimensional image of the internal structures. Acquisition of the projection images is time-consuming, and delivery of a treatment beam must be delayed during such acquisition. Also, additional systems, such as surface markers detectable in the projection images and by cameras in the treatment room, are often required to register the three-dimensional image with a coordinate space of the system used to deliver the treatment beam.

In one conventional example described in U.S. Pat. Nos. 6,812,842 and 7,289,839, transponders are implanted within a patient. The transponders emit signals which are captured and analyzed to determine their locations with respect to a treatment beam delivery system. By implanting the transponders within an organ or tumor, such systems are able to determine the location and/or orientation of the organ/tumor with respect to the delivery system and to thereby determine whether or not the location/orientation corresponds to a treatment plan.

Conventional implanted transponders suffer from large size, high cost and poor electromagnetic field compatibility. These deficiencies may be addressed by implanted radionuclide sources, such as RealEye™ by Navotek™. One or more scintillation imagers are used to detect radiation emitted by such sources, and the locations of the sources are determined based on the radiation.

Conventional radionuclide sources exhibit a long half-life and may therefore impart a significant radiation dose to the volume in which they reside (which might not be a target). Moreover, such sources raise a host of concerns relating to the handling and storing of radioactive materials.

### SUMMARY

In order to address the foregoing, some embodiments provide transmission of an activation beam to one or more non-radioactive elements disposed in a patient volume, wherein the elements become radioactive in response to the activation beam, detection of radiation emitted by the one or more now-radioactive elements, determination of respective locations of the one or more now-radioactive elements based on the detected radiation, and transmission of a treatment beam to the patient volume based on the respective locations.

Transmission of the treatment beam may include determination of whether the respective locations conform to a treatment plan, transmission of the treatment beam to the patient volume if it is determined that the respective locations conform to the treatment plan, and repositioning of the patient volume with respect to a treatment beam transmitter if it is determined that the respective locations do not conform to the treatment plan.

According to some aspects, a system includes one or more non-radioactive elements disposed in a patient volume, an activation beam transmitter to transmit an activation beam to the one or more non-radioactive elements, wherein the elements become radioactive in response to the activation beam, and one or more radiation detectors to detect radiation emitted by the one or more now-radioactive elements. A processor is to determine respective locations of the one or more now-radioactive elements based on the detected radiation, and a treatment beam transmitter is to transmit a treatment beam to the patient volume based on the respective locations.

The appended claims are not limited to the disclosed embodiments, however, as those in the art can readily adapt the descriptions herein to create other embodiments and applications.

### BRIEF DESCRIPTION OF THE DRAWINGS

Embodiments will become readily apparent from consideration of the following specification as illustrated in the accompanying drawings, in which like reference numerals designate like parts, and wherein:

FIG. 1 is a perspective view of a treatment room according to some embodiments;

FIG. 2 is a flow diagram of process steps pursuant to some embodiments;

FIG. 3 illustrates a system according to some embodiments; and

FIG. 4 illustrates operation of a system according to some embodiments.

### DETAILED DESCRIPTION

The following description is provided to enable a person in the art to make and use some embodiments and sets forth the best mode contemplated by the inventor for carrying out some embodiments. Various modifications, however, will remain readily apparent to those in the art.

As a brief introduction to the implementations described below, embodiments may concern the use of implantable non-radioactive elements to verify target position before, during and/or after radiation treatment. The elements are activated by an appropriate activation beam (i.e., the characteristics of which depend on the composition of the elements) such that the elements become radioactive. Radiation thereby emitted by the elements is detected and their respective locations are determined based on the radiation. These locations may then be used to determine if a target is in an appropriate position for treatment. In some embodiments, if the target is not in an appropriate position, adjustments (e.g., to the target position, to the gantry position, to the treatment beam) are determined based on the locations.

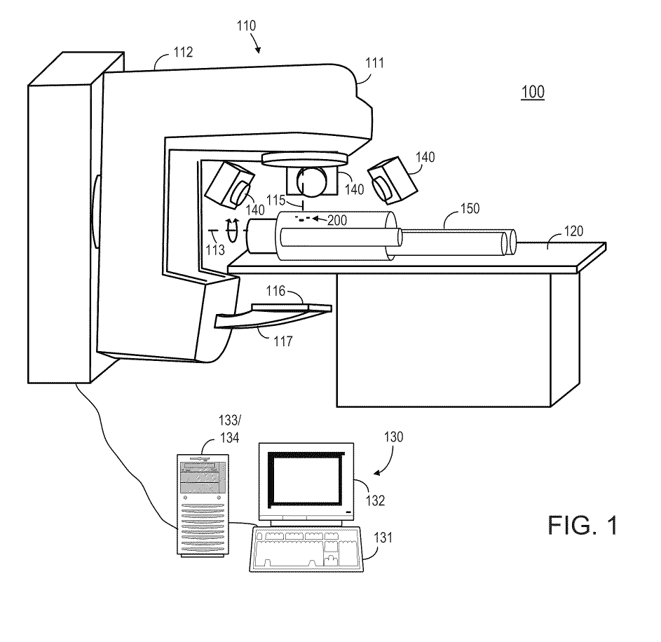

FIG. 1 is a perspective view of treatment room 100 according to some embodiments. Embodiments are not limited to the illustrated elements or arrangement thereof.

Radiation treatment room 100 includes linear accelerator (linac) 110, table 120, operator console 130 and detectors 140. The elements of radiation treatment room 100 may be used to deliver radiation to a target volume of beam object 150. In this regard, beam object 150 may comprise a patient positioned to receive radiation according to a radiation treatment plan.

Non-radioactive elements 200 are implanted within a volume of beam object 150. Elements 200 may be positioned around an internal structure of interest (e.g., a tumor, an organ) such that the locations of elements 200 provide a proxy for the position of the internal structure. For purposes of the present discussion, a “non-radioactive” element poses a negligible radiation risk to surrounding tissue.

Elements 200 may be of identical or different sizes. In FIG. 1, the apparent differences in the sizes of elements 200 are intended to reflect the various depths at which elements 200 are located.

Each of elements 200 may be rendered radioactive by an appropriate activation beam. A “radioactive” element emits ionizing particles and radiation at a detectable rate. Each of elements 200 may comprise one or more materials in any physical configuration. Embodiments may employ any suitable combination of element composition/configuration and activation beam.

For example, one or more of elements 200 may comprise Gold, while the activation beam used to render these elements radioactive may comprise an 18 MV x-ray beam. Such a beam may produce a neutron fluence per Gray of photon dose of 9.11×10⁶ neutrons/cm²/Gy at a treatment isocenter. Gold foil (¹⁹⁷Au) irradiated by such an activation beam produces 411.8 keV photons. These photons are readily detectable by a scintillation counter or by gamma cameras suitable for high-energy photons (e.g., Compton cameras). Advantageously, the U.S. Food and Drug Administration approves of Gold for internal use in medical practice. Moreover, Gold elements 200 would appear clearly on conventional x-ray projection images.

In some embodiments, one or more of elements 200 comprises Indium (¹¹⁵In). Indium emits gamma rays in response to photon activation, and the corresponding nuclear decay transforms ¹¹⁵In to ^{115m}In and ^{113m}In. Indium also appears clearly in kV and MV x-ray projection images due to its density and atomic number (i.e., 7.31 g/cm³ and 49, respectively). Although Indium exhibits low toxicity, some embodiments employ Gold-covered Indium elements to further decrease toxicity. One or more of elements 200 may comprise a biocompatible housing including one or more of a resin, plastic, ceramic, glass, and other biocompatible material that is or becomes known.

In some embodiments, one or more of elements 200 comprises Dysprosium (¹⁶⁴Dy). Dysprosium emits gamma rays in response to neutron activation and has a particularly large cross section for such reactions. Dysprosium also appears clearly in kilovoltage and megavoltage x-ray projection images due to its density and atomic number (i.e., 8.54 g/cm³ and 66, respectively). It is also a very effective contrast agent in magnetic resonance images, which allows implanted Dy elements 200 to be clearly identified in such images. The natural abundance of the isotope Dy-164 is 28.2%. Its thermal neutron cross section of 2,700 barns makes it one of the highest thermal neutron capture elements. When a thermal neutron is absorbed by Dy-164, a gamma-ray of approximately 100 keV energy is emitted. These can be detected using a standard gamma camera such as an Anger camera.

Absorption of a thermal neutron by Dy-164 also causes the emission of Beta particles. The dose induced by such particles can be accounted for by a treatment planning system and included in the therapeutic dose. In some embodiments, Dysprosium is enclosed in a material such as plastic or a “plastic scintillator” that is substantially transparent to gamma rays (50 keV-2 MeV) emitted as described above, but which substantially attenuates Beta particles having energies up to the MeV range. By reducing an additional dose due to the beta decay, clinical acceptance of an implanted element including Dy-164 may be more forthcoming. Although Dysprosium exhibits low toxicity, some embodiments employ Gold-covered Dysprosium elements to further decrease toxicity.

In some embodiments, a substance such as water is placed in the beam before it enters the tissue to slow down fast neutrons. This increases the number of neutrons that will interact with the elements 200. In other embodiments, a substance that converts photons to neutrons is likewise placed in the beam before it enters the tissue. Such a substance may also be included in the composition of the elements 200.

Detectors 140 therefore comprise any devices suitable to detect radiation emitted by activated elements 200. Examples include, but are not limited to, scintillation imagers and gamma cameras. Based on the detected radiation, detectors 140 also provide information suitable for determining locations of each of elements 200 in three-dimensional space. By registering detectors 140 within the coordinate space of linac 110, some embodiments facilitate determination of the locations of each of elements 200 with respect to a treatment isocenter. A treatment planning system may then determine whether these locations comply with a treatment plan.

Linac 110 generates and emits the radiation, and is primarily composed of treatment head 111 and gantry 112. Treatment head 111 includes a beam-emitting device (not shown) for emitting one or more radiation beams during treatment, calibration, and/or other scenarios. An emitted radiation beam may comprise electron, photon or any other type of radiation. According to some embodiments, the radiation beam exhibits energies in the megavoltage range (i.e. >1 MV) and may therefore be referred to as megavoltage radiation. Also included within treatment head 111 is a beam-shielding device, or collimator (not shown) for shaping the beam and for shielding sensitive surfaces from the beam.

Treatment head 111 is coupled to a projection of gantry 112. Gantry 112 is rotatable around gantry axis 113 before, during and after radiation treatment. Rotation of gantry 112 serves to rotate treatment head 111 around axis 113.

During radiation treatment, a radiation beam is emitted from treatment head 111 as a divergent beam. The beam is emitted towards an isocenter of linac 110. The isocenter is located at the intersection of beam axis 115 and gantry axis 113. Due to divergence of the radiation beam and the shaping of the beam by the aforementioned beam-shaping devices, the beam may deliver radiation to a volume of beam object 150 rather than only to the isocenter.

Table 120 supports beam object 150 during radiation treatment. Table 120 may be adjustable to assist in positioning a treatment area of beam object 150 at the isocenter of linac 110. Table 120 may also be used to support devices used for such positioning, for calibration and/or for verification.

Imaging device 116 may acquire images before, during and/or after radiation treatment. For example, imaging device 116 may be used to acquire images for verification and recordation of a target volume position and of an internal patient portal to which radiation is delivered.

Imaging device 116 may be attached to gantry 112 in any manner, including via extendible and retractable housing 117. Rotation of gantry 112 may cause treatment head 111 and imaging device 116 to rotate around the isocenter such that isocenter remains located between treatment head 111 and imaging device 116 during the rotation.

Imaging device 116 may comprise any system to acquire an image based on received megavoltage photon radiation. In a case that linac 110 is capable of producing kilovoltage photon radiation via beamline modification or other techniques, imaging device 116 may also acquire images based on such kilovoltage radiation. In some embodiments, imaging device 116 is a flat-panel imaging device using a scintillator layer and solid-state amorphous silicon photodiodes deployed in a two-dimensional array. In operation, the scintillator layer receives photons and generates light in proportion to the intensity of the received photons. The array of photodiodes receives the light and records the intensity of received light as stored electrical charge.

In other embodiments, imaging device 116 converts received photons to electrical charge without requiring a scintillator layer. The photons are absorbed directly by an array of amorphous selenium photoconductors. The photoconductors convert the photons directly to stored electrical charge. Imaging device 116 may also comprise a CCD or tube-based camera. Such an imaging device may include a light-proof housing within which are disposed a scintillator, a mirror, and a camera.

The charge developed and stored by imaging device 116 represents radiation intensities at each location of a radiation field produced by a beam emitted from treatment head 111. Since object 150 is located between treatment head and imaging device 116, the radiation intensity at a particular location represents the attenuative properties of tissues along a divergent line between a radiation source in treatment head 111 and the particular location. The set of radiation intensities acquired by imaging device 116 may therefore comprise a two-dimensional projection image of these tissues.

Operator console 130 includes input device 131 for receiving instructions from an operator and output device 132, which may be a monitor for presenting operational parameters of linac 110 and imaging device 116 and/or interfaces for receiving instructions. Output device 132 may also present a two-dimensional projection image, a three-dimensional megavoltage (or kilovoltage) cone beam image and/or two-dimensional “slice” images based on the three-dimensional image.

Input device 131 and output device 132 are coupled to processor 133 and storage 134. Processor 133 may execute program code to perform any of the functions described herein, and/or to cause linac 110 to perform any of the process steps described herein. For example, processor 133 may execute program code to determine locations of elements 200 based on signals received from detectors 140.

Storage 134 may store program code to generate and execute a treatment plan according to some embodiments. Such code may comprise the SyngoRT™ suite or the KONRAD™ treatment planning system sold by Siemens Medical Solutions. Accordingly, storage 134 may also store radiation treatment plans in accordance with any currently- or hereafter-known format. The treatment plans may comprise scripts that are automatically executable by elements of room 100 to provide radiation therapy fractions. Each fraction of each treatment plan may require a patient to be positioned in a particular manner with respect to treatment head 111.

Operator console 130 may be in a room other than treatment room 100, in order to protect its operator from radiation. For example, treatment room 100 may be heavily shielded, such as a concrete vault, to shield the operator from radiation generated by linac 110.

Linac 110 may be operated so that each emitted beam exhibits a desired intensity (e.g., represented in monitor units (MU)) and aperture (i.e., a cross-sectional shape determined at least in part by the above-mentioned collimator), and is delivered from a desired gantry angle. The intensity, aperture and gantry angle of a beam may be specified by a treatment plan, and control software may configure linac 110 to automatically execute such a treatment plan by automatically delivering beams of the desired intensities and shapes from the desired angles.

In addition to the foregoing characteristics, each beam may exhibit one of a plurality of beam energies provided by linac 110. For example, linac 110 may be capable of selectively delivering a 6 MV radiation beam or a 15 MV radiation beam, although embodiments are not limited to these two energies. Accordingly, each radiation beam delivered by linac 100 may be associated with an intensity, an aperture, an angle and one of two or more available energies. Linac 100 may therefore be used to deliver an appropriate activation bean to cause elements 200 to become radioactive, and to deliver a treatment beam according to a treatment plan.

In some embodiments, linac 110 includes features allowing rapid switching between two or more beam energies. The Siemens ONCOR Impression Plus linear accelerator includes an Auto Field Sequencer for switching between beam energies and may be suitable for use in conjunction with some embodiments.

According to some embodiments, the activation beam and the treatment beam are delivered by separate devices. For example, the activation beam may be delivered by a system capable of delivering beams having energies in the megavoltage range (i.e. >1 MV), while the treatment beam is delivered by a system capable of delivering beams having energies in the kilovoltage range (i.e. <1 MV).

A hardware environment according to some embodiments may include fewer or more elements than those shown in FIG. 1. For example, unshown elements may be required to provide AC power, RF power, cooling, hydraulics, vacuum and/or other systems needed to operate the illustrated elements. In addition, embodiments are not limited to the illustrated elements and/or environment.

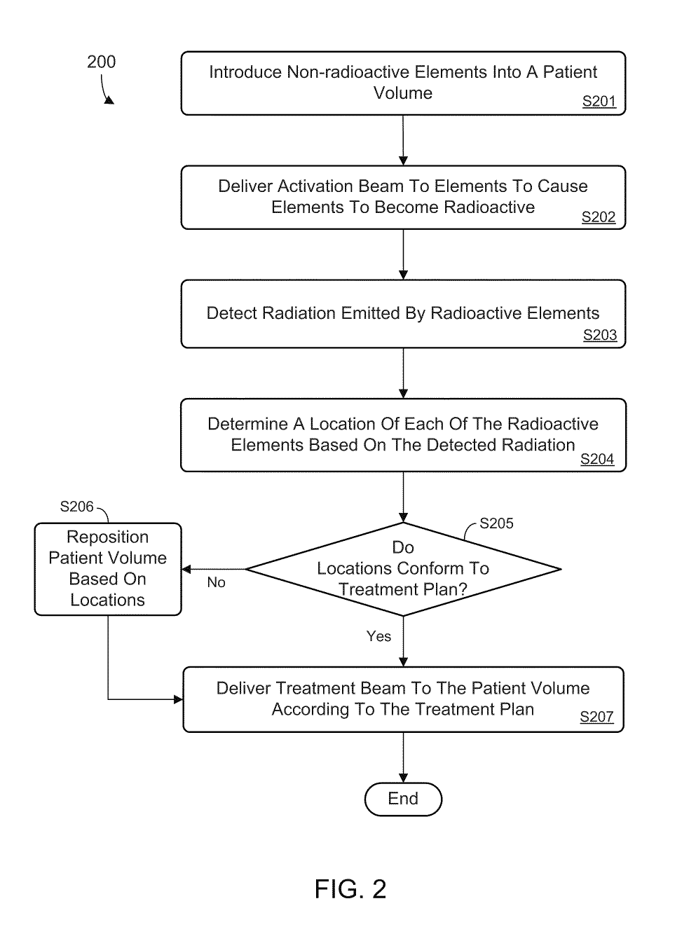

FIG. 2 is a flow diagram of a process according to some embodiments. Process 200 and the other processes described herein may be performed using any suitable combination of hardware, software or manual means. Software embodying these processes may be stored by any one or more tangible media, including but not limited to a fixed disk, a floppy disk, a CD-ROM, a DVD-ROM, a Zip™ disk, and a magnetic tape.

Prior to process 200, a treatment plan defining a target volume may be generated. The treatment plan is typically generated based on three-dimensional computed tomography images of the target volume and specifies treatment beam segments to be delivered to the target volume. Each segment is associated with a beam energy, a dose rate, a gantry position and a collimator configuration (i.e., a beam shape). Each segment may also require the target volume to be at a particular location and in a particular orientation with respect to the treatment isocenter.

Non-radioactive elements are introduced into a patient volume at S201. The elements may be introduced via an insertion tube (e.g., seeds), surgically, and/or by any other suitable system that is or becomes known. The elements may be introduced at locations which provide an indication of the location and orientation of the target volume.

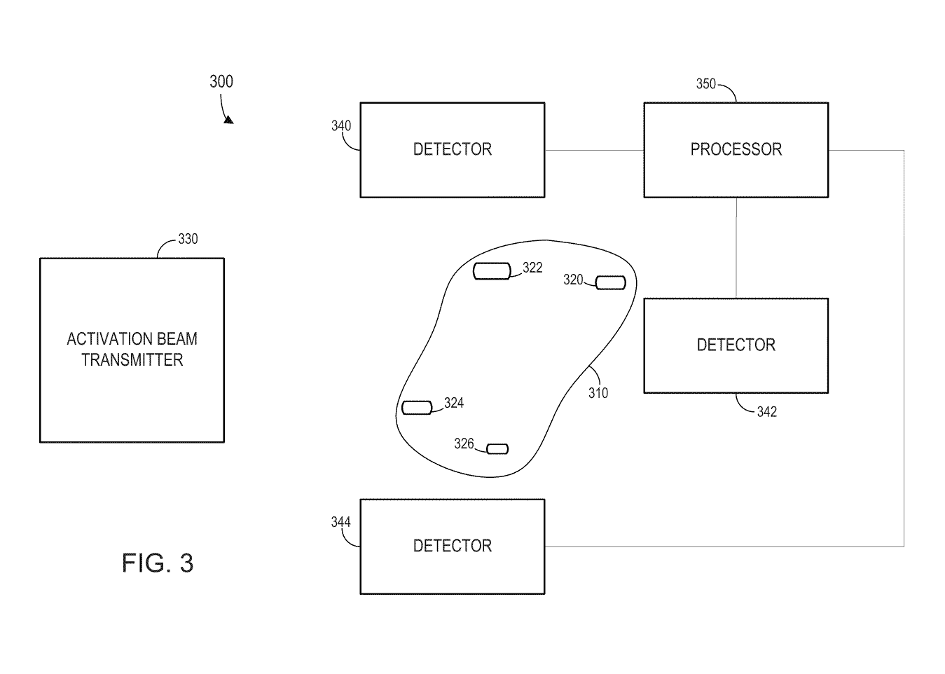

FIG. 3 illustrates system 300 to execute process 200 according to some embodiments. System 300 includes tumor (i.e., patient volume) 310, non-radioactive elements 320 through 326, activation beam transmitter 330, detectors 340 through 344 and processor 350.

Non-radioactive elements 320 through 326 may be positioned so as to provide a proxy for the location and orientation of tumor 310. The apparent differences in the sizes of elements 320 through 326 are intended to reflect the various depths at which elements 320 through 326 are located. Embodiments are not limited to the use of identically-sized non-radioactive elements.

Activation beam transmitter 330 may comprise linac 110 and/or a device separate from linac 110. Detectors 340 through 344 may comprise any of the radiation detectors described above, and need not be identical to one another. Processor 350 may comprise processor 134 of system 100 or a separate processor for performing process 200. For example, activation beam transmitter 330, detectors 340 through 344 and processor 350 may comprise elements of a system that is separable from linac 110 for use in other environments. Such a system may be sold and/or marketed separately from linac 110.

Returning to process 200, an activation beam is delivered to the elements at S202. As described above, and due to the composition of the activation beam and the elements, the elements become radioactive in response to the activation beam.

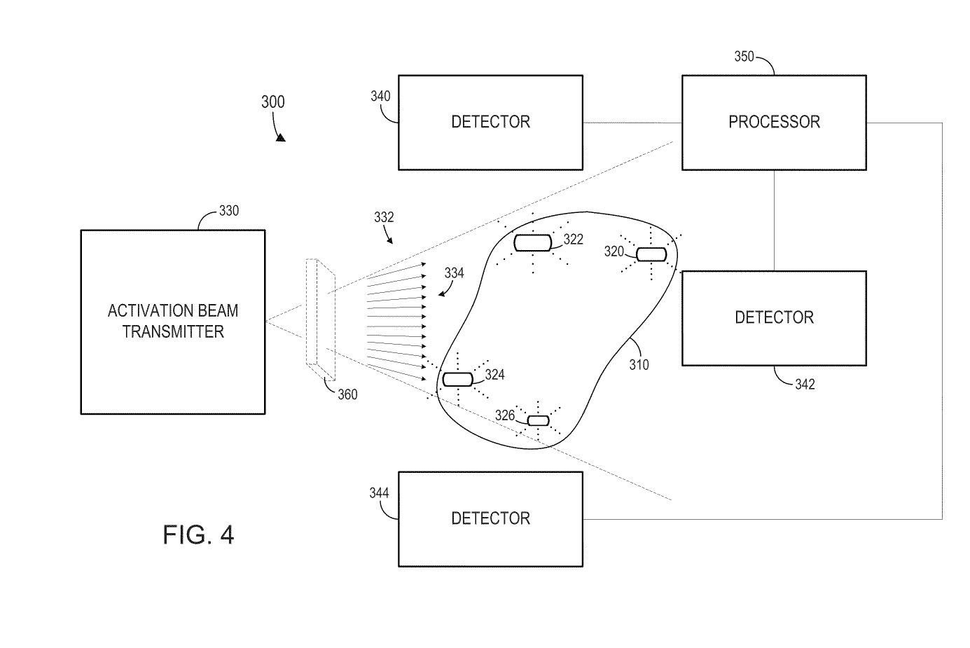

FIG. 4 illustrates system 300 of FIG. 3 during S202 according to some embodiments. Activation beam 332 intercepts each of elements 320 through 326, causing them to emit radiation. As mentioned above, the type of emitted radiation depends on the characteristics of activation beam 332 and the composition of elements 320 through 326.

Beam 332 includes flux 334 representing the particular particles of beam 332 which cause elements 320 through 326 to become radioactive. Flux 334 may comprise neutron flux or photon flux in some embodiments. The illustrated embodiment includes converter 360 for increasing the relevant flux of activation beam 332. In some embodiments, converter 360 comprises beryllium or water and operates to increase the neutron flux of activation beam 332.

Radiation emitted by the now-radioactive elements is detected at S203. Each of detectors 340 through 344 may, for example, detect all or a portion of the radiation emitted by elements 320 through 326. A location of each element is then determined at S204 based on the detected radiation. According to the present example, processor 350 receives data from detectors 340 through 344 and determines the locations based thereon. Any system may be used for the determination at S204, including but not limited to those used in the radiation-based systems described in the Background.

Next, at S205, it is determined whether the locations of the elements conform to the treatment plan. In this regard, the locations of elements 320 through 326 may serve as a proxy for the position (i.e., location and orientation) of tumor 310. Flow proceeds to S206 if this position fails to conform to the treatment plan. According to some embodiments, output device 132 may present an alert notifying an operator of a positioning error prior to proceeding to S206.

The patient volume is repositioned at S206 based on the determined locations. Repositioning of the patient volume may comprise moving one or more of table 120, patient 250 and gantry 112. That is, repositioning of the patient volume occurs with respect to the treatment isocenter, and does not necessarily involve moving the patient. Since both the desired position of the patient volume and the actual position of the patient volume are known (the latter via the locations of the elements), the difference between these positions may be used to inform movement of the patient volume.

In some embodiments, flow returns to S202 from S206 to check whether the movement(s) performed in S206 resulted in the desired conformity of the locations with the treatment plan. If it is determined at S205 that the locations conform to the treatment plan, flow proceeds to S207 for delivery of the treatment beam according to the treatment plan.

According to some embodiments, flow returns to S2021 after S207 and proceeds as described above after delivery of the treatment beam. Such an arrangement may provide position verification between treatment segments.

The several embodiments described herein are solely for the purpose of illustration. Therefore, persons in the art will recognize from this description that other embodiments may be practiced with various modifications and alterations.

What is claimed is:

1. A method comprising:
transmitting an activation beam to one or more non-radioactive elements disposed in a patient volume, wherein the elements become radioactive in response to the activation beam;
detecting radiation emitted by the one or more now-radioactive elements;
determining respective locations of the one or more now-radioactive elements based on the detected radiation; and
transmitting a treatment beam to the patient volume based on the respective locations.

2. A method according to claim 1, wherein the non-radioactive elements comprise Indium.

3. A method according to claim 1, wherein the non-radioactive elements comprise Gold.

4. A method according to claim 1, wherein the non-radioactive elements comprise Dysprosium.

5. A method according to claim 4, wherein each of the non-radioactive elements is enclosed in a material that is substantially transparent to gamma rays having energies in the 50 keV-2 MeV range, and which substantially attenuates Beta particles having energies up to the MeV range.

6. A method according to claim 1, wherein transmitting the activation beam to the one or more non-radioactive elements comprises:
transmitting the activation beam from an activation beam transmitter to a converter disposed between the activation beam transmitter and the one or more non-radioactive elements.

7. A method according to claim 1, wherein transmitting the treatment beam to the patient volume comprises:
determining whether the respective locations conform to a treatment plan;
transmitting the treatment beam to the patient volume if it is determined that the respective locations conform to the treatment plan; and
repositioning the patient volume with respect to a treatment beam transmitter if it is determined that the respective locations do not conform to the treatment plan.

8. A method according to claim 7, wherein repositioning the patient volume comprises:
repositioning the patient volume with respect to the treatment beam transmitter based on the respective locations.

9. A method according to claim 7, wherein transmitting the activation beam comprises:
transmitting the activation beam from the treatment beam transmitter.

10. A system comprising:
one or more non-radioactive elements disposed in a patient volume;
an activation beam transmitter to transmit an activation beam to the one or more non-radioactive elements, wherein the elements become radioactive in response to the activation beam;
one or more radiation detectors to detect radiation emitted by the one or more now-radioactive elements;
a processor to determine respective locations of the one or more now-radioactive elements based on the detected radiation; and
a treatment beam transmitter to transmit a treatment beam to the patient volume based on the respective locations.

11. A system according to claim 10, wherein the non-radioactive elements comprise Indium.

12. A system according to claim 10, wherein the non-radioactive elements comprise Gold.

13. A system according to claim 10, wherein the non-radioactive elements comprise Dysprosium.

14. A system according to claim 13, wherein each of the non-radioactive elements is enclosed in a material that is substantially transparent to gamma rays having energies in the 50 keV-2 MeV range, and which substantially attenuates Beta particles having energies up to the MeV range.

15. A system according to claim 10, further comprising:
a converter disposed between the activation beam transmitter and the one or more non-radioactive elements, the converter to increase neutron flux of the activation beam.

16. A system according to claim 10, wherein the processor is further to:
determine whether the respective locations conform to a treatment plan;
initiate transmission of the treatment beam from the treatment beam transmitter to the patient volume if it is determined that the respective locations conform to the treatment plan; and
initiate repositioning of the patient volume with respect to the treatment beam transmitter if it is determined that the respective locations do not conform to the treatment plan.

17. A system according to claim 16, wherein repositioning of the patient volume is based on the respective locations.

18. A system according to claim 10, wherein the activation beam transmitter and the treatment beam transmitter are the same.
